(19) United States
(12) Patent Application Publication
(10) Pub. No.: US 2025/0331892 A1
(43) Pub. Date: Oct. 30, 2025

(54) METHOD AND DEVICE FOR SUBCHONDRAL TREATMENT OF SPINE AND JOINTS

(71) Applicant: Le Reve Regenerative Wellness Inc., Campbell, CA (US)

(72) Inventors: Annu NAVANI, Campbell, CA (US); Raj NAVANI, Campbell, CA (US)

(73) Assignee: Le Reve Regenerative Wellness Inc., Campbell, CA (US)

(21) Appl. No.: 18/646,060

(22) Filed: Apr. 25, 2024

(51) Int. Cl. A61B 17/34; A61B 17/56; A61K 9/00; A61K 35/28

(52) U.S. Cl. CPC A61B 17/3472; A61B 17/3415; A61K 9/0019; A61K 35/28; A61B 2017/3454; A61B 2017/564

(57) ABSTRACT

Devices and methods for treating joint degeneration (e.g., intervertebral disc degeneration) are provided. An example method may comprise: (1) advancing a tip of an introducer needle into a nucleus pulpous of an intervertebral disc; (2) with the tip of the introducer needle in the nucleus pulpous, maneuvering, out of the tip of the introducer needle, a tip of a flexible needle into a region of a cartilaginous endplate (CEP) of the intervertebral disc; and (3) delivering, from an aperture of the flexible needle, therapeutic to the region of the CEP.

### BACKGROUND

Intervertebral discs are cartilaginous anatomical structures which separate vertebral bodies of the spinal column. The intervertebral discs act as shock absorbers that facilitate load transmission during spinal column movement, bending, twisting, etc.

An intervertebral disc comprises an outer fibrous structure called the annulus fibrosus that laterally surrounds an inner gel-like center called the nucleus pulposus (see e.g., FIG. 1). The intervertebral disc also comprises two cartilaginous end plates (CEPs) that separate the annulus fibrosus and nucleus pulposus from vertebral bodies the intervertebral disc is sandwiched between. Namely, the CEPs interface with subchondral endplates of the vertebral bodies. The subchondral endplates interface with cancellous bone of the vertebral bodies.

Sometimes, a CEP and a subchondral endplate are referred to collectively as a vertebral endplate. Here, the CEP would be referred to as a cartilaginous layer of the vertebral endplate that interfaces with the annulus fibrosus and nucleus pulposus of an intervertebral disc. The subchondral endplate would be referred to as a bony layer of the vertebral endplate that interfaces with cancellous bone of a vertebral body.

### BRIEF DESCRIPTION OF THE DRAWINGS

The present disclosure, in accordance with one or more various examples, is described in detail with reference to the following figures. The figures are provided for purposes of illustration only and merely depict example embodiments.

FIG. 1 illustrates a comparison between an example healthy intervertebral disc and an example intervertebral disc impacted by intervertebral disc degeneration (IDD), in accordance with various embodiments of the presently disclosed technology.

FIGS. 2A-2H illustrate how an example trocar-based device can deliver therapeutic to an IDD-impacted intervertebral disc and adjacent vertebral bodies, in accordance with various embodiments of the presently disclosed technology.

FIG. 3 depicts an example flexible needle of a trocar-based device, in accordance with various embodiments of the presently disclosed technology.

FIG. 4 depicts another example flexible needle of a trocar-based device, in accordance with various embodiments of the presently disclosed technology.

FIG. 5 depicts an example method for treating IDD, in accordance with various embodiments of the presently disclosed technology.

FIG. 6 depicts an example method for treating joint degeneration, in accordance with various embodiments of the presently disclosed technology.

FIG. 7 illustrates how an example trocar-based device can deliver therapeutic to a joint cavity and adjacent subchondral bone regions, in accordance with various embodiments of the presently disclosed technology.

The figures are not exhaustive and do not limit the present disclosure to the precise form disclosed.

### DETAILED DESCRIPTION

Lower back pain is a common cause of pain and dysfunction, especially in aging adults. Intervertebral disc degeneration (IDD) is a primary generator of lower back pain.

As the name suggests, IDD is a disease where intervertebral discs degenerate over time. For example, the nucleus pulposus will often change in cellular composition—becoming more fibrous and less elastic. Relatedly, collagen and other fibers in the annulus fibrosus can become disoriented and/or deteriorate. These compositional changes can impact an intervertebral disc's ability to function as an effective shock absorber. In many cases, the above-described degeneration can also reduce intervertebral disc height. Reduction in intervertebral disc height can cause spinal column narrowing and misalignment, and in some cases bone-on-bone contact between vertebral bodies.

Due in part to the compositional/morphological changes to the nucleus pulposus and annulus fibrosus discussed above, treatment for IDD has generally focused on the nucleus pulposus and the annulus fibrosus. For example, various treatments involve delivering therapeutic to the nucleus pulposus and annulus fibrosus in order to slow/prevent cell damage. Examples of such therapeutics include growth factors, autologous or allogeneic cell therapies, and other therapeutic agents.

While treatment for IDD has historically focused on the nucleus pulposus and the annulus fibrosus, recent research suggests that cellular changes to CEPs also play a role in the progression of IDD. Recent research has also indicated that cellular changes in the CEPs can result in structural/morphological changes in subchondral regions adjacent the CEPs, which can advance the progression of IDD and cause related issues. Such research—along with developments in treating knee osteoarthritis by delivering therapeutic to subchondral bone regions—suggests that IDD treatment can be improved by delivering therapeutic to CEPs and subchondral bone regions along with the nucleus pulposus and the annulus fibrosus.

However, delivering therapeutic to all these regions presents a serious challenge. Limited in part by existing needle and trocar designs, existing treatments would typically involve/require three separate needle insertions into a patient to deliver therapeutic to an intervertebral disc and the CEPs/subchondral regions superior and inferior to the intervertebral disc. For example, an existing treatment approach may involve making: (1) a first insertion (i.e., a first breaking of the skin and subsequent trajectory through tissue) to deliver therapeutic to the nucleus pulposus and annulus fibrosus of an intervertebral disc; (2) a second insertion (i.e., a second breaking of the skin and subsequent trajectory through tissue and bone) to deliver therapeutic to a superior (i.e., top) CEP and a subchondral region of a superior vertebral body adjacent the superior CEP; and (3) a third insertion (i.e., a third breaking of the skin and subsequent trajectory through tissue and bone) to deliver therapeutic to an inferior (i.e., bottom) CEP and a subchondral region of an inferior vertebral body adjacent the inferior CEP. Here, the second and third insertions typically involve advancing/drilling through bone of the superior and inferior vertebral bodies. Because of the bone drilling involved for the second and third insertions, these insertions would generally involve/require a different needle/trocar than the first insertion where a thinner diameter needle may be preferred to limit trauma to the intervertebral disc.

A concern with the existing treatment approach referenced above is that the additional needle insertions for delivering therapeutic to the superior and inferior CEPs/subchondral regions can increase patient trauma and surgical risk. Relatedly, advancing a needle through bone of vertebral bodies to access the CEPs/subchondral regions can also increase patient trauma and surgical risk, especially in patients with osteoporosis.

Embodiments of the disclosed technology improve upon existing treatments for IDD by providing a new device and method for delivering therapeutic to an intervertebral disc and subchondral regions of vertebral bodies adjacent the vertebral disc. The new device and method can deliver therapeutic to these three regions with just a single insertion into the intervertebral disc.

Relying on fewer insertions into a patient than existing/alternative treatments, embodiments can reduce patient trauma and surgical risk. Relatedly, by accessing CEPs and subchondral regions of adjacent vertebral bodies from the intervertebral disc—as opposed to accessing these regions by advancing/drilling through bone of the vertebral bodies—embodiments can further reduce patient trauma and surgical risk.

For example, embodiments provide a new trocar-based based device with structural features specially adapted for this new treatment method. Such features include: (1) a hollow introducer needle dimensioned to be inserted into an intervertebral disc; (2) a flexible needle that can be advanced through and out of a tip of the hollow introducer needler once the hollow introducer needler is in place within the intervertebral disc; and (3) a handle at a distal end of the trocar-based device that can be manipulated by a clinician to maneuver the flexible needle (e.g., with 360 degree rotation) to different regions within or proximate to the intervertebral disc.

The hollow introducer needle may comprise a cylindrical cavity running the length of the hollow introducer needle. Accordingly, a smaller diameter needle (e.g., the flexible needle referenced above) or probe can be advanced through this cylindrical cavity to eventually exit a distal end of the cylindrical cavity (corresponding with the “tip” of the hollow introducer needle).

The hollow introducer needler may have a tailored/particularized diameter (e.g., 22 gauge-25 gauge) that reduces trauma to the intervertebral disc (facilitated by a relatively smaller diameter) while still being sufficiently wide to allow a flexible needle thick/sturdy enough to make incisions into bone to pass through it. The hollow introducer needle may also have a length (e.g., 3.5 inches-6 inches) tailored/particularized to allow a tip of the hollow introducer needle to be approximately centrally located within the intervertebral disc after insertion.

The flexible needle may be fluidly connected to a reservoir of therapeutic such that the therapeutic can be delivered via the flexible needle. For example, a clinician can depress a plunger that is mechanically connected to the reservoir such that depressing of the plunger pushes the therapeutic out one or more apertures proximate a tip of the flexible needle. The flexible needle may have a sharp tip (e.g., a Quinke tip or a Chiba tip) that can cut through bone in order to deliver therapeutic to subchondral regions of vertebral bodies adjacent the intervertebral disc. In certain embodiments, the flexible needle may comprise a micro-needle array (e.g., proximate the tip of the flexible needle) that delivers the therapeutic to multiple anatomical locations—thus increasing treatment efficacy and efficiency. The flexible needle may comprise a sturdy material that can be flexed along a curved trajectory—such as stainless steel or titanium.

As alluded to above, the handle may be located at a proximal end of the trocar-based device (i.e., an end of the trocar-based device opposite the tip of the introduced needle). The handle may be mechanically connected to the flexible needle to allow a clinician to maneuver the flexible needle by manipulating the handle. In certain implementations, the handle may allow for 360 degree rotation of the flexible needle.

As alluded to above, the presently disclosed trocar-based device can facilitate a new method for treating IDD that involves fewer insertions into a patient, and less advancing/drilling through bone. For example, the method may involve advancing a tip of a hollow introducer needle of the trocar-based device into a patient's intervertebral disc. During such advancement, a stopper probe may be inserted within the cylindrical cavity of the hollow introducer needle to prevent tissue from entering the cylindrical cavity. With the hollow introducer needle in place within the intervertebral disc, the stopper probe can be removed, and a flexible needle can be advanced through the cylindrical cavity of the hollow introducer needle in the stopper probe's place. As alluded to above, the flexible needle may be fluidly connected to a reservoir of therapeutic. Accordingly, the therapeutic can be delivered to different regions within or proximate to the intervertebral disc by maneuvering a tip of the flexible needle to the different regions.

For example, with the tip of the introducer needle in the nucleus pulpous, the tip of the flexible needle can be maneuvered out of the tip of the introducer needle and into a region of a superior CEP of the intervertebral disc. Accordingly, the therapeutic can be delivered to the region of the superior CEP from the flexible needle (e.g., via one or more apertures proximate the tip of the flexible needle).

In certain implementations, after delivering the therapeutic to the region of the superior CEP, the tip of the flexible needle can be advanced through the superior CEP and into a subchondral region of a vertebral body superior to and adjacent the intervertebral disc. Accordingly, the therapeutic can be delivered to the subchondral region of the superior vertebral body. As alluded to above, the flexible needle may have a sharp tip (e.g., a Quincke tip or a Chiba tip) that can cut through bone in order to enter the subchondral region of the superior vertebral body. The flexible needle may also have a particularized length dimension (e.g., between 11 and 16 inches) that allows the flexible needle to reach the subchondral region of the superior vertebral body via the intervertebral disc.

In some implementations, after delivering the therapeutic to the region of the superior CEP (or to the subchondral region of the superior vertebral body), the tip of the flexible needle can be retracted back to the nucleus pulpous. From this position, the tip of the flexible needle can then be maneuvered to a second region of the superior CEP to deliver therapeutic to the second region of the superior CEP. Alternatively, the tip of the flexible needle can be maneuvered to a region of an inferior CEP of the intervertebral disc to deliver therapeutic to the region of the inferior CEP. In some of these implementations, after delivering the therapeutic to the region of the inferior CEP, the flexible needle can be advanced through the inferior CEP and into a subchondral region of a vertebral body inferior to and adjacent the intervertebral disc. Accordingly, the therapeutic can be delivered to the subchondral region of the inferior vertebral body as well.

It should be understood that the above-described methodology/order is merely an example. For instance, the flexible needle can be maneuvered to deliver therapeutic to the inferior CEP/inferior vertebral body before the superior CEP/superior vertebral body. Relatedly, the flexible needle can be maneuvered to regions of the nucleus pulposus and the annulus fibrosus to deliver the therapeutic to those anatomical regions as well.

Moreover, the principles disclosed herein may be extended beyond IDD therapies. For example, devices and methods of the presently disclosed technology may be used to treat other joints such as knees, shoulders, hips, elbows, etc.

Embodiments of the presently disclosed technology are now described in greater detail in conjunction with the following FIGs.

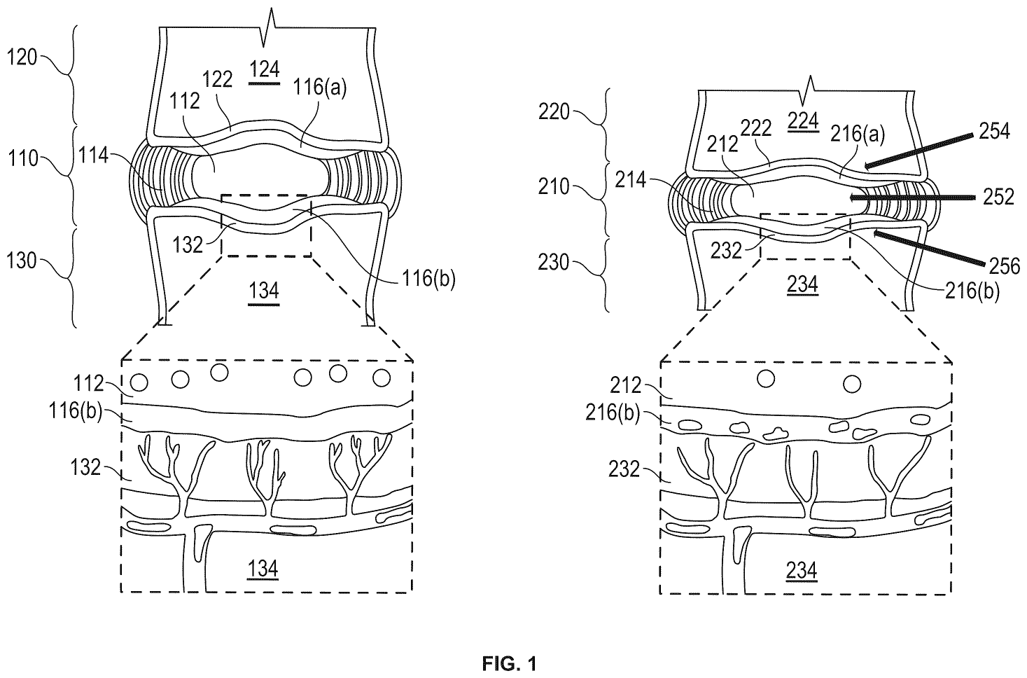

FIG. 1 illustrates a comparison between an example healthy intervertebral disc 110 and an example intervertebral disc 210 impacted by IDD, in accordance with various embodiments of the presently disclosed technology.

As depicted, healthy intervertebral disc 110 comprises a nucleus pulposus 112 and an annulus fibrosus 114. Annulus fibrosus 114 comprises an outer fibrous anatomical structure that laterally surrounds nucleus pulposus 112. Nucleus pulpous 112 may comprise a gel-like anatomical structure that facilitates load transmission during spinal column movement, bending, twisting, etc.

Healthy intervertebral disc 110 also comprises a superior cartilaginous end plate (CEP) 116(a) and an inferior CEP 116(b).

Superior CEP 116(a) may comprise a cartilaginous anatomical structure that separates nucleus pulpous 112 and portions of annulus fibrosus 114 from superior vertebral body 120. Namely, superior CEP 116(a) interfaces with a subchondral endplate 122 of superior vertebral body 120. Subchondral endplate 122 may interface with a cancellous bone region 124 of superior vertebral body 120.

Similar to superior CEP 116(a), inferior CEP 116(b) may comprise a cartilaginous anatomical structure that separates nucleus pulpous 112 and portions of annulus fibrosus 114 from inferior vertebral body 130. Namely, inferior CEP 116(b) interfaces with a subchondral endplate 132 of inferior vertebral body 130. Subchondral endplate 132 may interface with a cancellous bone region 134 of inferior vertebral body 130.

As alluded to above, sometimes a CEP and a subchondral endplate are referred to collectively as a vertebral endplate. For example, superior CEP 116(a) and subchondral endplate 122 may be referred to as a first (superior) vertebral endplate. Likewise, inferior CEP 116(b) and subchondral endplate 132 may be referred to as a second (inferior) vertebral endplate.

As depicted, blood vessels can supply blood to regions of superior vertebral body 120 and inferior vertebral body 130—including subchondral endplate 122 and subchondral endplate 132. Such blood flow can mitigate the impacts of cell degeneration in these bone regions.

Referring now to IDD-impacted intervertebral disc 210, IDD-impacted intervertebral disc 210 comprises a nucleus pulposus 212 and an annulus fibrosus 214. As described above, IDD can cause nucleus pulposus 212 to change in cellular composition—becoming more fibrous and less elastic. Relatedly, collagen and other fibers in annulus fibrosus 214 can become disoriented and/or deteriorate. These compositional changes can impact IDD-impacted intervertebral disc 210's ability to function as an effective shock absorber. As depicted, the above-described degeneration can also result in height reduction for IDD-impacted intervertebral disc 210. Such height reduction cause spinal column narrowing and misalignment, and in some cases bone-on-bone contact between superior and inferior vertebral bodies 220 and 230 respectively. Height reduction for IDD-impacted intervertebral disc 210 may also cause structural/morphological changes to superior and inferior vertebral bodies 220 and 230.

IDD-impacted intervertebral disc 210 also comprises a superior CEP 216(a) and an inferior CEP 216(b). As depicted, IDD can cause cellular changes to these CEPs that can result in calcification and/or other morphological changes. As depicted, calcification (and/or other morphological changes) to superior CEP 216(a) and an inferior CEP 216(b) can cause structural/morphological changes to superior vertebral body 220 and inferior vertebral body 230 respectively. For example, calcification (and/or other morphological changes) to superior CEP 216(a) can cause reduced vascular connections and reduced blood flow in subchondral endplate 222 and cancellous bone region 224. Relatedly, calcification (and/or other morphological changes) to inferior CEP 216(b) can cause reduced vascular connections and reduced blood flow in subchondral endplate 232 and cancellous bone region 234. Reductions in vascular connections and blood flow can negatively impact the health of these bone regions.

As described above (and as depicted in FIG. 1), the impacts of IDD can extend beyond the nucleus pulposus and annulus fibrosus—to CEPs and subchondral regions of adjacent vertebral bodies. Accordingly, treatment for IDD can be improved by delivering therapeutic to these anatomical regions as well.

However, delivering therapeutic to all these regions presents a serious challenge. Limited in part by existing needle and trocar designs, existing treatments would typically involve three separate insertions into a patient to deliver therapeutic to IDD-impacted intervertebral disc 210 and the CEPs/subchondral regions superior and inferior to IDD-impacted intervertebral disc 210. For example, an existing treatment approach may involve making: (1) a first insertion (represented by trajectory 252) to deliver therapeutic to nucleus pulposus 212 and annulus fibrosus 214; (2) a second insertion (represented by trajectory 254) to deliver therapeutic to superior CEP 216(a) and subchondral regions superior vertebral body 220; and (3) a third insertion (represented by trajectory 256) to deliver therapeutic to inferior CEP 216(b) and subchondral regions of inferior vertebral body 230. Here, the second and third needle insertions (represented by trajectory 254 and trajectory 256 respectively) typically involve advancing/drilling through bone of superior vertebral body 220 and inferior vertebral body 230. Because of the bone drilling involved for the second and third insertions, these insertions would generally involve/require a different needle/trocar than the first insertion where a thinner diameter needle may be preferred to limit trauma to the intervertebral disc.

A concern with this existing treatment approach is that the additional needle insertions for delivering therapeutic to the superior and inferior CEPs/subchondral regions can increase patient trauma and surgical risk. Relatedly, advancing a needle through bone of vertebral bodies to access the CEPs/subchondral regions can also increase patient trauma and surgical risk, especially in patients with osteoporosis.

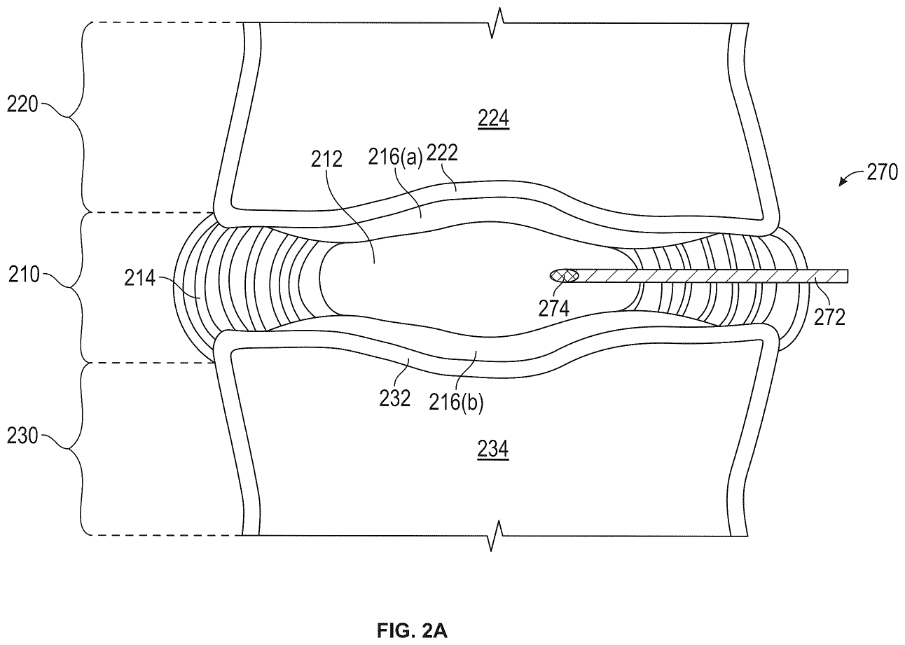

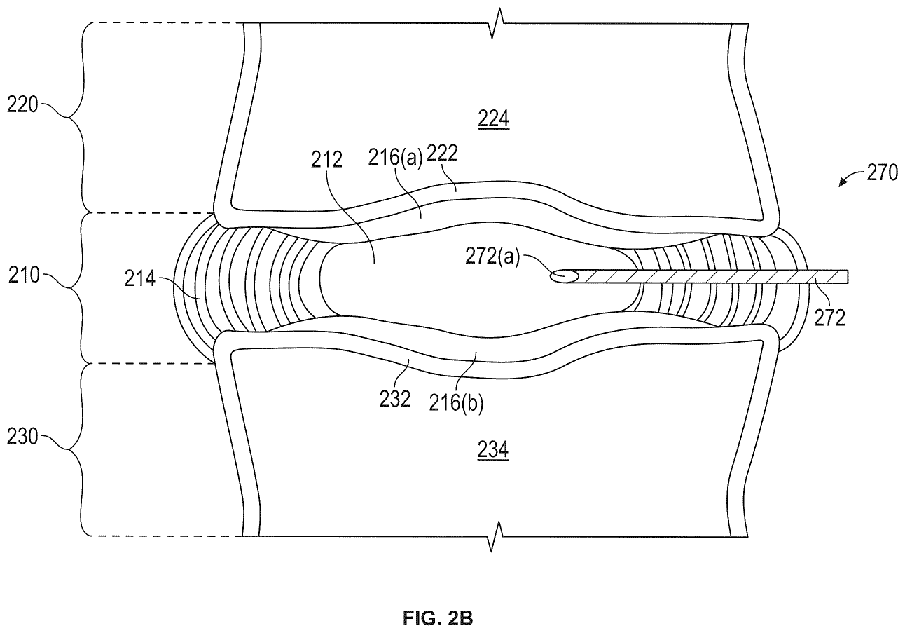

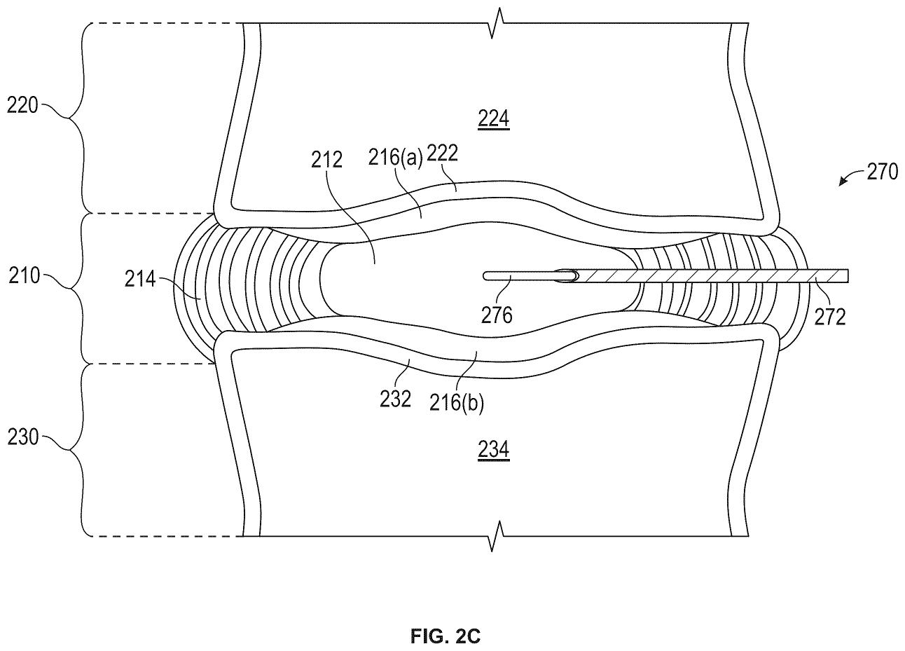

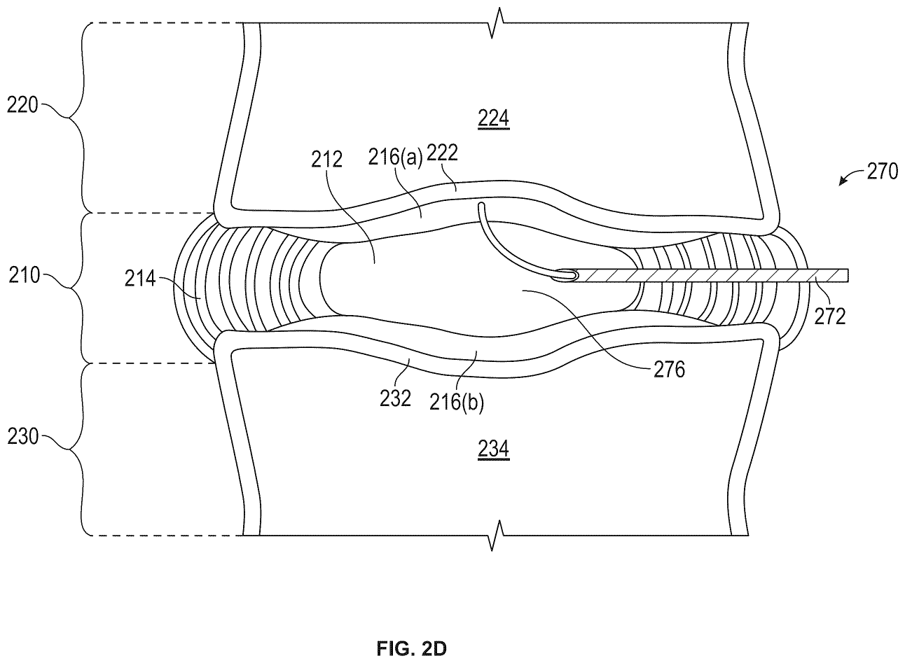

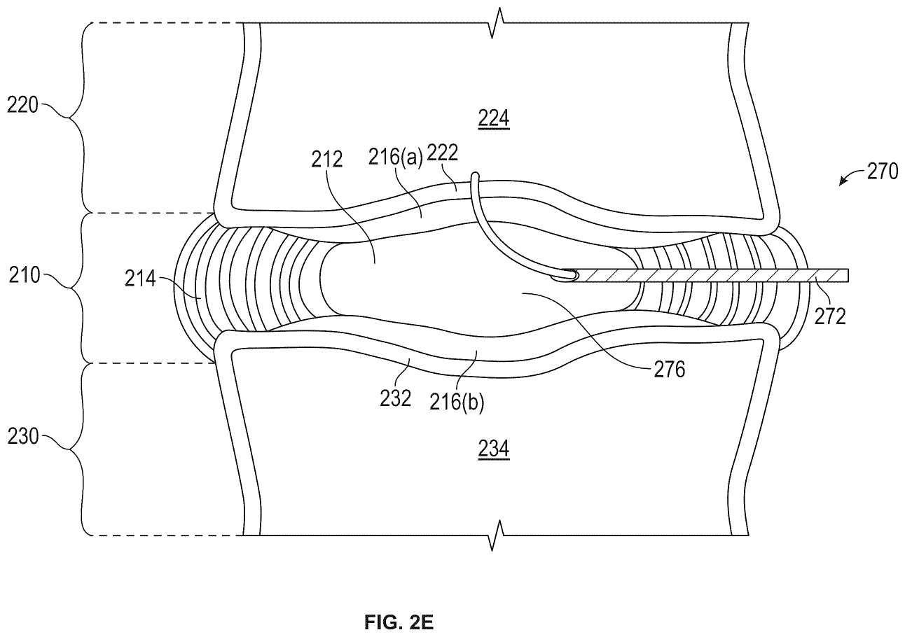

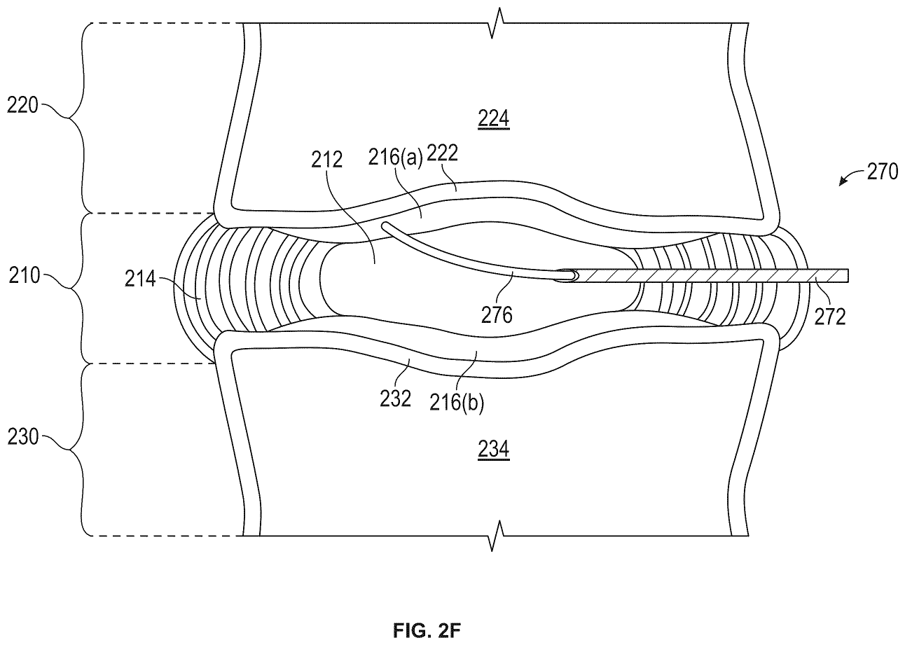

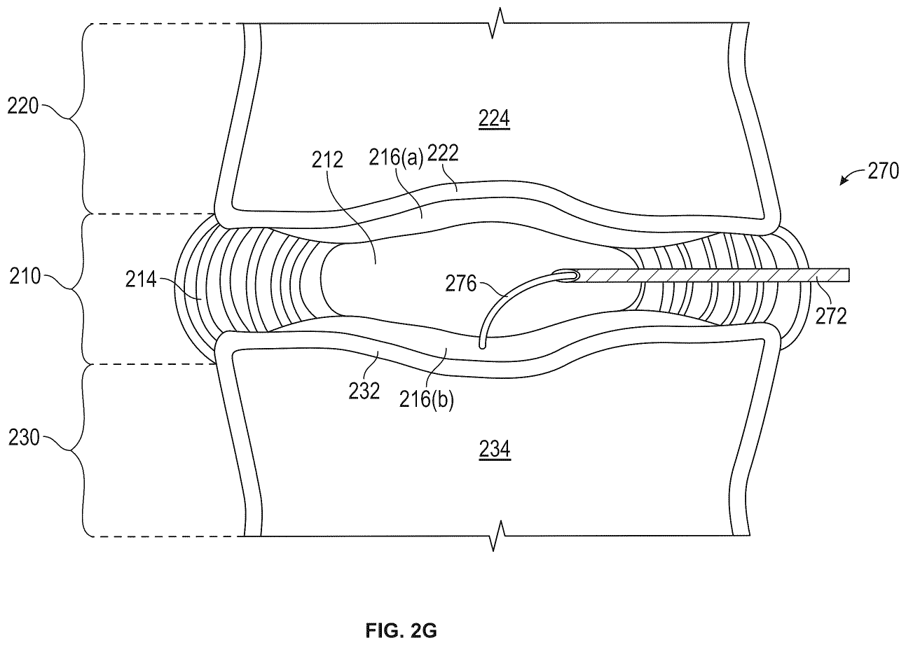

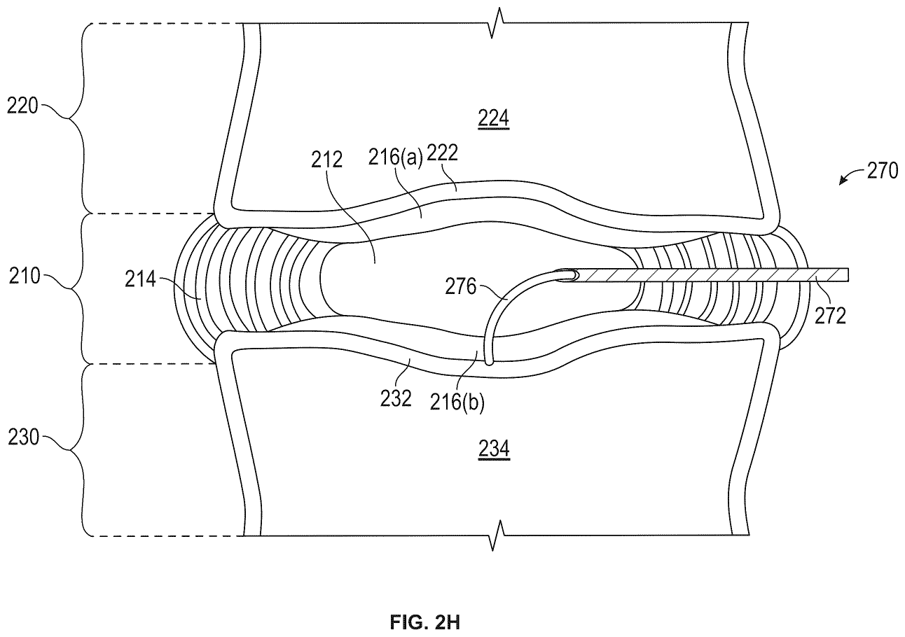

As described above, embodiments of the disclosed technology improve upon existing treatments for IDD by providing a new device and method for delivering therapeutic to an intervertebral disc and subchondral regions of vertebral bodies adjacent the vertebral disc. The new device and method can deliver therapeutic to these three regions with just a single insertion into the intervertebral disc. Such a device and method are described in greater detail in conjunction with FIGS. 2A-2H.

FIGS. 2A-2H illustrate how an example trocar-based device 270 can deliver therapeutic to IDD-impacted intervertebral disc 210 and adjacent vertebral bodies, in accordance with various embodiments of the presently disclosed technology.

As depicted, trocar-based device 270 may comprise a hollow introducer needle 272. Hollow introducer needle 272 may have a cylindrical cavity 272(a) (depicted in FIG. 2B) running the length of hollow introducer needle 272. In other words, a proximal end (i.e., right-side end in FIGS. 2A-2H) of cylindrical cavity 272(a) may correspond with a proximal end of hollow introducer needle 272. Likewise, a distal end (i.e., left-side end in FIGS. 2A-2H) of cylindrical cavity 272(a) may correspond with a distal end of hollow introducer needle 272. Herein, the distal end of hollow introducer needle 272 is sometimes referred to as a tip of hollow introducer needle 272.

A probe (e.g., stopping probe 274 depicted in FIG. 2A) or needle (e.g., flexible needle 276 depicted in FIGS. 2C-2H) can be inserted into cylindrical cavity 272(a) through a proximal end of cylindrical cavity 272(a) (corresponding with a proximal end of hollow introducer needle 272). In various implementations, the proximal end of cylindrical cavity 272(a) (corresponding with the proximal end of hollow introducer needle 272) may remain outside patient tissue during a surgical intervention.

After being inserted into/through the proximal end of cylindrical cavity 272(a), the probe or needle can be advanced through cylindrical cavity 272(a). In certain implementations, the probe or needle can eventually be advanced out a distal end of cylindrical cavity 272(a) (corresponding with a distal end, or “tip,” of hollow introducer needle 272). For example, and as depicted in FIGS. 2C-2H, flexible needle 276 can be advanced out a distal end of cylindrical cavity 272(a). As the distal end of cylindrical cavity 272(a) corresponds with a tip of hollow introducer needle 272, flexible needle 276 is sometimes referred to as being advanced out a tip of hollow introducer needle 272.

As alluded to above, hollow introducer needle 272 can be particularly dimensioned for insertion into an intervertebral disc, such as IDD-impacted intervertebral disc 210. For example, hollow introducer needler 272 may have a tailored/particularized diameter (e.g., 22 gauge-25 gauge) that reduces trauma to IDD-impacted intervertebral disc 210 (facilitated by a relatively smaller diameter) while still being sufficiently wide to allow a flexible needle (e.g., flexible needle 276 depicted in FIGS. 2C-2H) thick/sturdy enough to make incisions into bone to pass through it. Hollow introducer needle 272 may also have a length (e.g., e.g., 3.5 inches-6 inches) tailored/particularized to allow a tip (i.e., distal end) of hollow introducer needle 272 to be approximately centrally located within IDD-impacted intervertebral disc 210 after insertion. Hollow introducer needle 272 may comprises various types of materials, such as titanium, stainless steel, etc. In certain embodiments, the walls of hollow introducer needle 272 (i.e., the difference between inner and outer diameter of hollow introducer needle 272) may be between 0.1 mm and 0.3 mm. It should be understood that the size of hollow introducer needle 272 relative to IDD-impacted intervertebral disc 210 in FIGS. 2A-2H does not limit or depict an actual size relationship. Instead, these drawings are sized to illustrate structural and methodological features, without implying relative size relationships.

As depicted in FIG. 2A, hollow introducer needle 272 can be advanced into IDD-impacted intervertebral disc 210. For example, hollow introducer needle 272 can first be advanced into and through annulus fibrosus 214 and then into nucleus pulposus 212. This can be achieved without advancing hollow introducer needle 272 through bone (e.g., through superior vertebral body 220 and/or inferior vertebral body 230). By avoiding advancing hollow introducer needle 272 through bone, embodiments can reduce patient trauma and surgical risk—especially in patients with osteoporosis. Relatedly, because hollow introducer needle 272 is not advanced through bone, it may enable a relatively smaller diameter for hollow introducer needle 272 that reduces trauma to IDD-impacted intervertebral disc 210.

As depicted in FIG. 2A, in certain implementations a stopper probe 274 can be inserted within cylindrical cavity 272(a) when hollow introducer needle 272 is being advanced through/into IDD-impacted intervertebral disc 210. With stopping probe 274 filling cylindrical cavity 272(a) during advancement, stopping probe 274 can prevent tissue from entering cylindrical cavity 272(a)/hollow introducer needle 272. As depicted in FIG. 2B, when hollow introducer needle 272 is in a desired position within IDD-impacted intervertebral disc 210 for deploying flexible needle 276 (depicted in FIGS. 2C-2H), stopper probe 274 can be removed from cylindrical cavity 272(a) by retracting stopper probe 274 out the proximal end of cylindrical cavity 272(a)/proximal end of hollow introducer needle 272.

As depicted in FIG. 2C, after stopper probe 274 has been removed from cylindrical cavity 272(a), flexible needle 276 can be advanced through cylindrical cavity 272(a)—and eventually exit the distal end of cylindrical cavity 272(a) (corresponding with a tip of hollow introducer needle 272).

As alluded to above, flexible needle 276 may be fluidly connected to a reservoir of therapeutic such that the therapeutic can be delivered via flexible needle 276. For example, a clinician can depress a plunger that is mechanically connected to the reservoir such that depressing of the plunger pushes the therapeutic out one or more apertures proximate a tip/distal end of flexible needle 276. The therapeutic may comprise various types of therapeutics such as an autologous cell-based therapeutic (e.g., autologous bone marrow concentrate (BMC)), an allogenic cell-based therapeutic, a growth factor-based therapeutic, acellular therapeutics, chemical substances like local anesthetics and steroids, and other types of therapeutics. In certain implementations, flexible needle 276 may comprise heating coil for delivering heat therapy. In some implementations, flexible needle 276 may deliver electric impulses for electric stimulation therapy. In various implementations, flexible needle 276 may be used for ionizing therapy or radiation therapy.

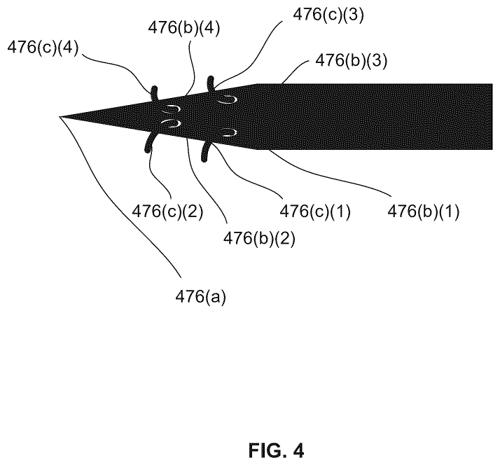

Flexible needle 276 may have a sharp tip (e.g., a Quinke tip or a Chiba tip) that can cut through bone in order to deliver therapeutic to subchondral regions of superior vertebral body 220 and inferior vertebral body 230. In certain embodiments, flexible needle 276 may comprise a micro-needle array (described in greater detail in conjunction with FIG. 4) proximate the tip of flexible needle 276 that delivers the therapeutic to multiple locations. Flexible needle 276 may comprise a sturdy material that can be flexed along a curved trajectory (see e.g., FIGS. 2D-2H)—such as stainless steel or titanium.

As alluded to above, trocar-based device 270 may further comprise a handle (not depicted) at a proximal end of trocar-based device 270. The handle may be mechanically connected to flexible needle 276 to allow a clinician to maneuver flexible needle 276 by manipulating the handle. In certain implementations, the handle may allow for 360 degree rotation of flexible needle 276. For example, a physician can rotate the handle to turn/curve flexible needle 276 in the direction of rotation. This may happen when advancing or retracting flexible needle 276. For example, turning the handle right may curve the tip of flexible needle 276 to the right, and vice versa.

As depicted in FIG. 2C, with the tip of flexible needle 276 within nucleus pulposus 212, therapeutic can be delivered to nucleus pulposus 212 via one or more apertures proximate the tip of flexible needle 276.

As depicted in FIG. 2D, with hollow introducer needle 272 in the same/similar position within nucleus pulposus 212, the tip of flexible needle 276 can be advanced/maneuvered (e.g., along a curved trajectory) into a region of a superior CEP 216(a). Accordingly, therapeutic can be delivered to the region of the superior CEP 216(a) via the one or more apertures proximate the tip of flexible needle 276.

As depicted in FIG. 2E, the tip of flexible needle 276 can be (further) advanced through superior CEP 216(a) and into a subchondral region of superior vertebral body 220. In some implementations, such further advancement may be performed after delivering therapeutic to the region of the superior CEP 216(a).

The subchondral region of superior vertebral body 220 may comprise one or more of a region of subchondral endplate 222 and a region of cancellous bone region 224 adjacent subchondral endplate 222. With the tip of flexible needle 276 within the subchondral region of superior vertebral body 220, therapeutic can be delivered to the subchondral region of superior vertebral body 220.

As alluded to above, flexible needle 276 may have a sharp tip (e.g., a Quincke tip or a Chiba tip) that can cut through bone in order to enter the subchondral region of superior vertebral body 220. As alluded to above, having a relatively smaller diameter for flexible needle 276 can enable a relatively smaller diameter for hollow introducer needle 272. Having a relatively smaller diameter for these needles can reduce trauma to IDD-impacted intervertebral disc 210 during insertion/advancement.

As alluded to above, flexible needle 276 may also have a particularized length dimension (e.g., between 11 and 16 inches) that allows flexible needle 276 to reach the subchondral region of superior vertebral body 220 via IDD-impacted intervertebral disc 210.

As depicted in FIG. 2F, with hollow introducer needle 272 in the same/similar position within nucleus pulposus 212, the tip of flexible needle 276 can also be advanced/maneuvered (e.g., along a curved trajectory) into a second region of a superior CEP 216(a). Accordingly, therapeutic can be delivered to the second region of superior CEP 216(a) via the one or more apertures proximate the tip of flexible needle 276.

As depicted in FIG. 2G, with hollow introducer needle 272 in the same/similar position within nucleus pulposus 212, the tip of flexible needle 276 can also be advanced/maneuvered (e.g., along a curved trajectory) into a region of inferior CEP 216(b). Accordingly, therapeutic can be delivered to the region of inferior CEP 216(b) via the one or more apertures proximate the tip of flexible needle 276.

As depicted in FIG. 2H, the tip of flexible needle 276 can be (further) advanced through inferior CEP 216(b) and into a subchondral region of inferior vertebral body 230. In some implementations, this further advancement may be performed after delivering therapeutic to the region of inferior CEP 216(b).

The subchondral region of inferior vertebral body 230 may comprise one or more of a region of subchondral endplate 232 and a region of cancellous bone region 234 adjacent subchondral endplate 232. With the tip of flexible needle 276 within the subchondral region of inferior vertebral body 230, therapeutic can be delivered to the subchondral region of inferior vertebral body 230 as well.

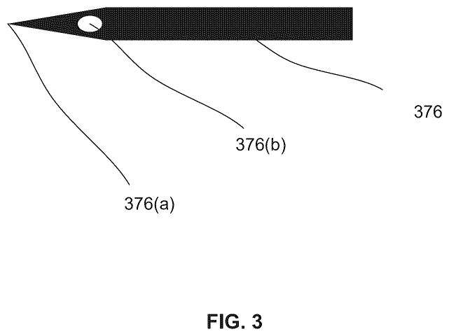

FIG. 3 depicts a zoomed-in view of an example flexible needle 376 of a trocar-based device, in accordance with various embodiments of the presently disclosed technology.

Here, flexible needle 376 may be an example implementation of flexible needle 276 from FIGS. 2A-2H.

As depicted, flexible needle 376 comprises a sharp tip 376(a) and an aperture 376(b) located proximate to sharp tip 376(a).

As alluded to above, sharp tip 376(a) may comprise a Quincke tip, a Chiba tip, or another type of sharp needle tip capable of cutting through small portions of bone.

Aperture 376(b) may be located proximate to sharp tip 376(a), such as within 0.1-0.5 mm of sharp tip 376(a).

As alluded to above, flexible needle 376 may be fluidly connected to a reservoir of therapeutic. Accordingly, therapeutic may be delivered by pushing therapeutic out of aperture 376(b).

In various implementations, flexible needle 376 may include additional apertures proximate sharp tip 376(a). Here, inclusion of additional apertures can allow therapeutic to be delivered to a larger anatomical area from a common position for flexible needle 376. In various implementations, flexible needle 376 may comprise a ring of two or more apertures (including aperture 376(b)) around a cylindrical/conical circumference of flexible needle 376 proximate sharp tip 376(a).

FIG. 4 depicts an example flexible needle 476 of a trocar-based device, in accordance with various embodiments of the presently disclosed technology.

Here, flexible needle 476 may be another example implementation of flexible needle 276 from FIGS. 2A-2H.

As depicted, flexible needle 476 comprises a sharp tip 476(a) and micro-needle array proximate sharp tip 476(a).

The micro-needle array comprises a region of flexible needle 476 that includes two or more apertures, such as aperture 476(b)(1), aperture 476(b)(2), aperture 476(b)(3), and aperture 476(b)(4).

As depicted, the micro-needle array also comprises two or more micro-needles which can be advanced through the length of flexible needle 476 and out respective apertures of the micro-needle array. For example, the micro-needle array includes a micro-needle 476(c)(1) which can be advanced out of aperture 476(b)(1). The micro-needle array also includes a micro-needle 476(c)(2) which can be advanced out of aperture 476(b)(2), a micro-needle 476(c)(3) which can be advanced out of aperture 476(b)(3), and a micro-needle 476(c)(4) which can be advanced out of aperture 476(b)(4). In should be understood that in various embodiments the micro-needle array may comprise a different number and configuration of apertures and micro-needles.

As depicted, in certain implementations the micro-needles of the micro-needle array can be advanced out their respective apertures along a curved trajectory.

In various implementations, each of the micro needles may comprise one or more of their own apertures proximate their respective tips. Relatedly, each micro-needle may be fluidly connected to a reservoir of therapeutic (either a common reservoir, or two or more separate reservoirs). Accordingly, the therapeutic can be delivered by the micro-needles by pushing the therapeutic out the apertures of the micro-needles.

As alluded to above, inclusion of the micro-needle array can allow therapeutic to be delivered to a larger anatomical area from a common position for flexible needle 476.

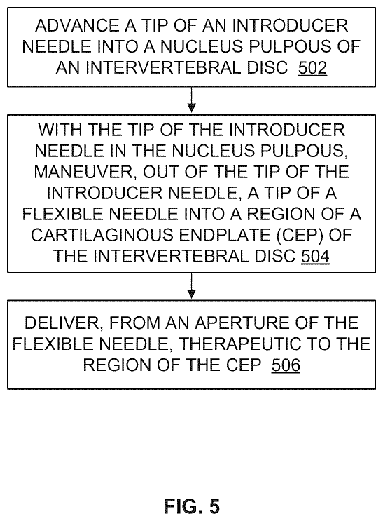

FIG. 5 depicts an example method for treating IDD, in accordance with various embodiments of the presently disclosed technology.

As depicted, at operation 502, embodiments can advance a tip of an introducer needle into a nucleus pulpous of an intervertebral disc.

At operation 504, with the tip of the introducer needle in the nucleus pulpous, embodiments can maneuver, out of the tip of the introducer needle, a tip of a flexible needle into a region of a cartilaginous endplate (CEP) of the intervertebral disc.

At operation 506, embodiments can deliver, from an aperture of the flexible needle, therapeutic to the region of the CEP.

In certain implementations, after delivering the therapeutic to the region of the CEP, embodiments can retract the tip of the flexible needle from the region of the CEP back to the nucleus pulpous. From the nucleus pulpous, embodiments can then maneuver the tip of the flexible needle into a second region of the CEP. Accordingly, embodiments can deliver, from the aperture of the flexible needle, therapeutic to the second region of the CEP.

In some implementations, after delivering the therapeutic to the region of the CEP, embodiments can similarly retract the tip of the flexible needle from the region of the CEP back to the nucleus pulpous. From the nucleus pulpous, embodiments can then maneuver the tip of the flexible needle into a region of a second CEP of the intervertebral body. Accordingly, embodiments can deliver, from the aperture of the flexible needle, therapeutic to the region of the second CEP

In various implementations, after delivering the therapeutic to the region of the CEP, embodiments can advance the tip of the flexible needle through the CEP and into a subchondral region of a vertebral body adjacent the intervertebral disc. Accordingly, embodiments can deliver, from an aperture of the flexible needle, therapeutic to the subchondral region of the vertebral body.

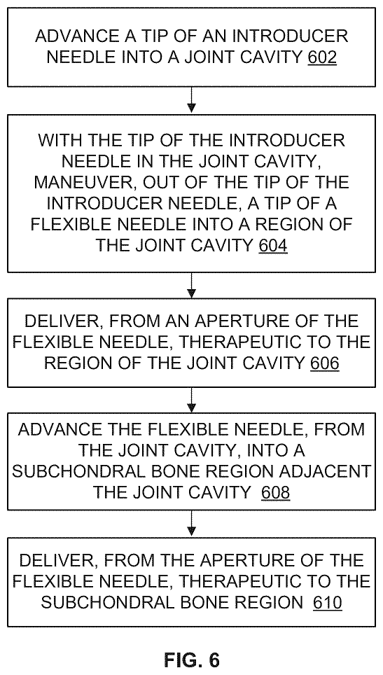

FIG. 6 depicts an example method for treating joint degeneration, in accordance with various embodiments of the presently disclosed technology.

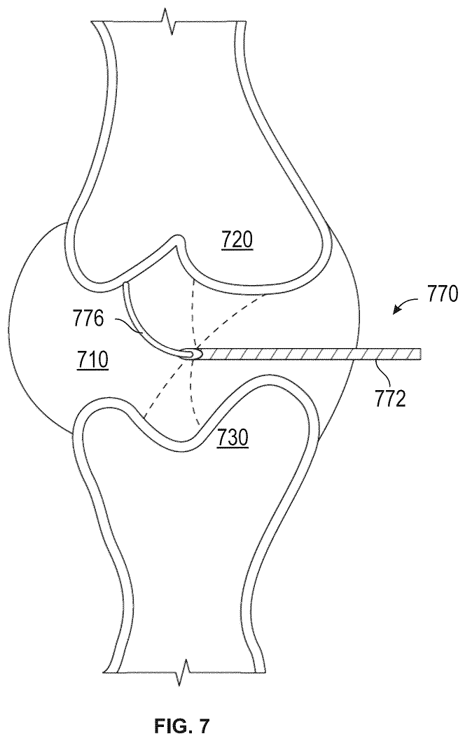

As a companion figure, FIG. 7 illustrates how an example trocar-based device 770 can deliver therapeutic to a joint cavity 710 and adjacent subchondral bone regions, in accordance with various embodiments of the presently disclosed technology.

As alluded to above, devices and methods of the presently disclosed technology may be used to treat other joints such as knees, shoulders, hips, elbows, etc.

For example, and as depicted in FIGS. 6 and 7, a trocar-based device 770 may be used to deliver therapeutic to a joint cavity 710 and a subchondral bone region adjacent the joint cavity (e.g., a subchondral region of bone 720).

For example, at operation 602, embodiments can advance a tip of an introducer needle 772 (of trocar-based device 770) into joint cavity 710. Such advancement may avoid cutting through bone adjacent joint cavity 710.

With the tip of introducer needle 772 in joint cavity 710, at operation 604 embodiments can maneuver, out of the tip of introducer needle 772, a tip of a flexible needle 776 into a region of joint cavity 710.

At operation 606, embodiments can deliver, from an aperture of flexible needle 776, therapeutic to the region of joint cavity 710.

Then, at operation 608, embodiments can advance flexible needle 776, from joint cavity 710, into a subchondral bone region adjacent the joint cavity (e.g., a subchondral region of bone 720 or a subchondral region of bone 730).

Accordingly, at operation 610, embodiments can deliver, from the aperture of flexible needle 776, therapeutic to the subchondral bone region.

It should be understood that the various features, aspects and functionality described in one or more of the individual embodiments are not limited in their applicability to the particular embodiment with which they are described. Instead, they can be applied, alone or in various combinations, to one or more other embodiments, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1. A method for treating intervertebral disc degeneration, the method comprising:
advancing a tip of an introducer needle into a nucleus pulpous of an intervertebral disc;
with the tip of the introducer needle in the nucleus pulpous, maneuvering, out of the tip of the introducer needle, a tip of a flexible needle into a region of a cartilaginous endplate (CEP) of the intervertebral disc; and
delivering, from an aperture of the flexible needle, therapeutic to the region of the CEP.

2. The method of claim 1, further comprising:
after delivering the therapeutic to the region of the CEP, retracting the tip of the flexible needle from the region of the CEP back to the nucleus pulpous;
from the nucleus pulpous, maneuvering the tip of the flexible needle into a second region of the CEP; and
delivering, from the aperture of the flexible needle, therapeutic to the second region of the CEP.

3. The method of claim 1, further comprising:
after delivering the therapeutic to the region of the CEP, retracting the tip of the flexible needle from the region of the CEP back to the nucleus pulpous;
from the nucleus pulpous, maneuvering the tip of the flexible needle into a region of a second CEP of the intervertebral body; and
delivering, from the aperture of the flexible needle, therapeutic to the region of the second CEP.

4. The method of claim 1, further comprising:
after delivering the therapeutic to the region of the CEP, advancing the tip of the flexible needle through the CEP and into a subchondral region of a vertebral body adjacent the intervertebral disc; and
delivering, from an aperture of the flexible needle, therapeutic to the subchondral region of the vertebral body.

5. The method of claim 1, further comprising:
advancing the tip of the introducer needle into an annulus fibrosus of the intervertebral disc;
wherein advancing the tip of the introducer needle into the nucleus pulpous of the intervertebral disc is from the annulus fibrosus of the intervertebral disc.

6. The method of claim 1, wherein:
the introducer needle and the flexible needle are components of a trocar-based device; and
maneuvering the tip of the flexible needle comprises manipulating a handle of the trocar-based device at a distal end of the trocar-based device opposite the tip of the introducer needler.

7. The method of claim 6, wherein the handle is manipulatable to facilitate 360 degree rotation of the flexible needle.

8. The method of claim 1, wherein the therapeutic comprises at least one of:
an autologous cell-based therapeutic;
an allogenic cell-based therapeutic; and
a growth factor-based therapeutic.

9. The method of claim 1, wherein the therapeutic comprises autologous bone marrow concentrate (BMC).

10. The method of claim 1, wherein the tip of the flexible needle comprises a micro-needle array that delivers the therapeutic to multiple locations of the region of the CEP.

11. The method of claim 1, wherein the tip of the flexible needle comprises a Quinke tip or a Chiba tip.

12. The method of claim 1, wherein diameter of the introducer needle is between 21 gauge and 26 gauge.

13. The method of claim 1, wherein a length of the flexible needle is between 11 and 16 inches.

14. The method of claim 1, wherein the flexible needle comprises at least one of stainless steel and titanium.

15. A method for treating intervertebral disc degeneration, the method comprising:
advancing a tip of an introducer needle into a nucleus pulpous of an intervertebral disc;
with the tip of the introducer needle in the nucleus pulpous, maneuvering, out of the tip of the introducer needle, a tip of a flexible needle into a region of a cartilaginous endplate (CEP) of the intervertebral disc;
delivering, from an aperture of the flexible needle, therapeutic to the region of the CEP;
after delivering the therapeutic to the region of the CEP, advancing the tip of the flexible needle through the CEP and into a subchondral region of a vertebral body adjacent the intervertebral disc; and
delivering, from an aperture of the flexible needle, the therapeutic to the subchondral region of the vertebral body.

16. The method of claim 15, further comprising:
after delivering the therapeutic to the subchondral region of the vertebral body, retracting the tip of the flexible needle from the subchondral region of the vertebral body;
from the nucleus pulpous, maneuvering the tip of the flexible needle into a second region of the CEP; and
delivering, from the aperture of the flexible needle, therapeutic to the second region of the CEP.

17. The method of claim 15, wherein the tip of the flexible needle comprises a Quinke tip or a Chiba tip.

18. The method of claim 15, wherein diameter of the introducer needle is between 22 gauge and 25 gauge.

19. The method of claim 15, wherein the therapeutic comprises autologous bone marrow concentrate (BMC).

20. A method for treating joint degeneration, the method comprising:
advancing a tip of an introducer needle into a joint cavity;
with the tip of the introducer needler in the joint cavity, maneuvering, out of the tip of the introducer needle, a tip of a flexible needle into a region of the joint cavity;
delivering, from an aperture of the flexible needle, therapeutic to the region of the joint cavity;
advancing the flexible needle, from the joint cavity, into a subchondral bone region adjacent the joint cavity; and
delivering, from the aperture of the flexible needle, therapeutic to the subchondral bone region.
